(19) United States
(12) Patent Application Publication
(10) Pub. No.: US 2006/0204645 A1
(43) Pub. Date: Sep. 14, 2006

(54) Method of coating a surgical instrument

(76) Inventor: Herman P. Godfried, Amsterdam (NL)

Correspondence Address: Jenkens & Gilchrist;a Professional Corporation, Suite 3700, 1445 Ross Avenue, Dallas, TX, 75202, US

(21) Appl. No.: 11/312,044

(22) Filed: Dec. 20, 2005

Related U.S. Application Data
(63) Continuation-in-part of application No. 10/048,131, filed on Aug. 1, 2002, now abandoned.

(30) Foreign Application Priority Data
Jul. 30, 1999 (SA) SA 99/4910

(51) Int. Cl. A61L 33/00

(52) U.S. Cl. 427/2.1

(57) ABSTRACT

A method of forming a protective layer of fluorine atoms on a cutting blade of a surgical instrument in which the blade is formed of a hard, transparent, crystalline material such as diamond, sapphire or garnet. According to the method the blade is placed in a plasma reactor, the blade is then plasma cleaned and coated with a plasma of carbon fluoride gas. A method of forming a protective layer of fluorine atoms on a blade for surgical instruments in which the blade is immersed into a solution of fluoroaliphatic silyl ether.

This application is a continuation-in-part of U.S. patent application Ser. No. 10/048,131, filed Jan. 23, 2002, which was the National Stage of International Application No. PCT/IB 00/01066, filed Jul. 31, 2000.

### BACKGROUND OF THE INVENTION

This invention relates to a method of coating a surface of a surgical instrument.

Surgical blades are extremely sharp in order to minimise tissue damage along a line of incision. In order to achieve the desired sharpness of a cutting blade, ordinary materials such as surgical grade stainless steel blades may not have the required properties and materials of choice for the manufacture of such cutting blades are hard materials of a crystalline nature, such as diamond or sapphire.

During use, blood and other bodily fluids and materials often stick to the facets of a cutting blade, thereby reducing its effectiveness. It is well known that in order to prevent this from happening, or at least reduce the sticking effect and facilitate cleaning of the blade, wiping the blade with a suitable material or sticking it into a block of suitable plastic foam, for example polystyrene, may be necessary.

The problem of blood sticking to or coagulating on the surface of a cutting blade may be aggravated under conditions where coagulation of blood is promoted. This may be caused by deliberate heating of the surgical blade to induce coagulation; by high intensity light sources used in conjunction with the blade or by the simultaneous use of a laser beam, either through the cutting blade or applied separately.

South African provisional patent application no. 99/4256, also filed by the applicant in this instance, describes a cutting blade for a surgical instrument in which the cutting blade is formed of diamond and laser radiation is transmitted through the blade in order to provide a cauterisation effect along a line of incision. This earlier application is incorporated herein by reference. The laser radiation passing through the cutting blade which forms the subject of this invention would cause heating of the blade which encourages blood sticking and coagulating on the surface of the blade.

### SUMMARY OF THE INVENTION

According to a first aspect of the invention there is provided a method of forming a protective layer of fluorine atoms on a cutting blade of a surgical instrument in which the blade is formed of hard, transparent, crystalline material, such as diamond, sapphire or garnet, the method comprising the steps of:

a) placing the blade in a plasma reactor;

b) plasma cleaning the blade; and

c) coating the blade in a plasma of carbon fluoride (CₙFₘ) gas.

Preferably, the carbon fluoride (CₙFₘ) containing gas is C₃F₈, alternatively C₂F₄ or C₂F₆.

The method may include the step of chemically cleaning the blade.

Typically, the coating takes place at a pressure of 0.01 to 2 mbar, for a period of 30 to 180 minutes and at a power level of 50 to 2000 watts.

Conveniently, the cleaning takes place in a plasma of air, oxygen, argon or a mixture thereof.

According to a second aspect of the invention there is provided a cutting blade for a surgical instrument, the cutting blade being formed of a hard, transparent, crystalline material, such as diamond, sapphire or garnet, on the surface of which is provided a protective layer of fluorine atoms formed in accordance with the method described above.

Preferably, the blade is formed of natural, monocrystalline synthetic or polycrystalline synthetic diamond or sapphire.

According to a third aspect of the invention there is provided a method of forming a protective layer of fluorine atoms on a surface of a surgical instrument characterised in that the method comprises the step of immersing the blade into a solution of a fluoroaliphatic silyl ether.

The method of the third aspect of the invention is typically performed on a surface formed of diamond.

Preferably, the method of the third aspect of the invention includes the step of curing the layer at a temperature in excess of 200° C.

The method of the third aspect of the invention may include a step of forming a hydroxyl terminated surface on the blade before immersion of the blade into a solution of a fluoroaliphatic silyl ether.

The method may also include the step of forming an intermediate silicon or Ti layer on the surface of the surface prior to immersion of the blade into a solution of a fluoroaliphatic silyl ether. The Si layer preferably has a thickness less than 50 nm.

The surgical instrument may be suitable for a single or one-off application such as a window through which laser or like radiation passes, in use. It is a surface of the window which is coated by the method of the third aspect of the invention. The surgical instrument may also be suitable for multiple applications such as a knife. It is a surface of the cutting blade of the knife which is coated by the method of the third aspect of the invention.

The thickness of the protective coating is preferably no more than a few hundred nanometres, e.g. no more than 700 nanometres.

Various embodiments of the invention are described in detail in the following passages of the specification. The described embodiments are merely illustrative of how the invention might be put into effect and should not be seen as limiting on the scope of the invention.

### DESCRIPTION OF AN EMBODIMENT

In general terms various embodiments of the invention relate to a method of forming a protective layer of fluorine atoms on a cutting blade for a surgical instrument in which the surgical blade is formed of a hard, transparent, crystalline material such as diamond, sapphire or garnet. The purpose of the layer is to reduce the sticking effect of blood and bodily fluids and materials to the blade during use. The layer should be of minimum thickness to minimise the reduction in sharpness of the blade. It is envisaged that this may be achieved according to the invention either by minimising the thickness of the layer (in the extreme case one atomic layer of fluorine) or by polishing a micro facet on one or both sides of the cutting edge after the coating has been applied.

The method of various embodiments of the invention is in essence a plasma coating method involving the following steps:
1. Chemically cleaning the blade.
2. Placing the cutting blade in a plasma reactor.
3. Plasma cleaning of the blade. This is done in a plasma of air, oxygen, argon or a mixture thereof for 5 to 20 minutes at approximately 1 mbar pressure and a power level of approximately 500 watts. The power is switched on at a duty cycle of 5% to 50% to prevent overheating. This cleaning step is essential if good adhesion of the fluorine containing layer is to be achieved.
4. Coating the blade in a plasma of C₃F₈. The process conditions of this coating step are a pressure of 0.01 to 2 mbar for a period of 30 to 180 minutes at a power level between 50 and 2000 watts.

The above description is a description of one method of putting the process of the invention into effect and of variations on the specific process conditions described above.

Two different approaches may be used in the process described above:
1. The chemical structure of the diamond or other hard, crystalline material is modified such that it terminates with fluorine atoms, instead of the more usual hydrogen and/or oxygen. This can be achieved by exposing the surface of the material, such as diamond, to atomic fluorine at a range of temperatures, between 273 and 573K. The preferred deposition method for the fluorine atomic layer onto the surgical blade is plasma treatment. In this method the surgical blade is exposed to a plasma excited in an atomic fluor generating substance such as SF₆, NF₃, HF or F₂. Argon may be introduced into the plasma to reduce the deposition rate to controllable levels.
2. The surface is coated with a fluorocarbon polymer layer. This can be achieved by the known technique of plasma polymerization using precursors such as tetrafluoroethene. This process is described in the article entitled “Fundamentals of Plasma Chemistry and Technology” H. V. Boenig, Pub Technomatic, 1988 and the other references referred to in this document, which are all incorporated herein by reference.
   The preferred deposition method for the fluorocarbon polymer layer onto the surgical blade is plasma treatment. In this method the surgical blade is exposed to a plasma excited in a carbon fluoride gas. Argon may be introduced into the plasma to reduce the deposition rate to controllable levels.
   The thickness of the fluorocarbon polymer layer created by this process is a function of the time for which the blade is subjected to the process. The coating thickness can vary from a few nanometers to hundreds of nanometers. Thinner coatings are more desirable so as not to blunt the cutting edge of the blade and limit laser light absorption.
   The polymer is deposited from a plasma excited from one of the following gases:
   C₂F₄, C₂F₆, C₃F₈.
   The layer thickness is typically between 5 nanometers and 10 microns. A micro facet of between 5 and 50 microns is polished on one or both sides of the cutting edge after the layer has been formed.

In addition to the methods described above other processes may also be used to achieve the desired layer of fluorine atoms on the surface. One such method is to heat the blade in a C₂F₄ environment. This induces polymerisation of the C₂F₄ on the hot surfaces to form a layer of fluorine atoms.

Various embodiments of the invention also provide a method of applying a coating of fluorine atoms on a surface of a surgical instrument, in which method the fluorine atoms may be chemically bonded to the surface by attaching a chemically reactive group to a fluorinated alkane group. Such a fluorinated alkane is a molecule in which fluorine atoms replace hydrogen atoms in a (usually linear) carbon chain. This is an inert molecule and a polymerised variant is the basis for the product known by the proprietary name of “Teflon”. By attaching a chemically reactive group to the fluorinated alkane it can be bonded to the diamond surface. An example of such a chemically reactive group is a group containing SiOH, which can bond to a surface, which is hydroxyl (—OH) terminated. The SiOH group can bond to the hydroxyl terminated surface by splitting off a water molecule, thus forming a fluorinated_tail-Si—O—Si-surface bond. An example of this type of coating material is fluoroaliphatic silyl ethers, whose generic chemical formula is given below.
RfA—Si(OH)3

A schematic representation of this reaction is provided as follows: 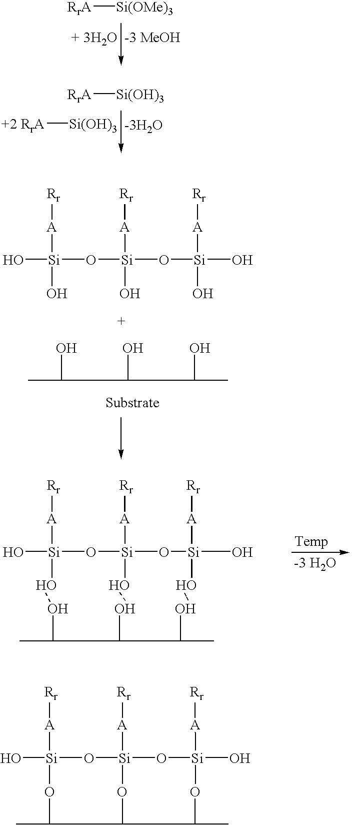
where Rf is a fluorinated alkyl group, A is C₂H₄, and Si(OH)₃ is the active bonding group. In this case one of the OH groups can bond to the surface, while the others bond to other fluoroaliphatic silyl ether molecules, thus forming a network.

An example of a fluoroaliphatic silyl ether is the product sold under the brand name FC405/60 by the 3M company. Here the fluoroaliphatic silyl ether molecules are dissolved in a solvent such as an alcohol (e.g. isopropanol). By further diluting the solution with isopropanol so that a concentration of the fluoroaliphatic silyl ether molecules is obtained of less than 1% (e.g. adding 0.5 ml of coating fluid to 60 ml of isopropanol) and adding acetic acid to give a value of the pH of between 4 and 5.5, a layer of fluorine atoms can be applied to the surface of a diamond blade by dipping it in the solution for approximately 3 minutes. It is recommended that the solution be stirred ultrasonically to establish good contact of fresh coating fluid with the surface. The surface is drawn out of the coating fluid and the remaining layer of coating solution is rinsed off with isopropanol. The coating is then allowed to cure at an elevated temperature. Although the product information supplied by the manufacturer of the fluoroaliphatic silyl ether fluid states that curing should take place for 5 minutes at 110° C., it has been found that a coating with better scratch and rubbing resistance and better adherence to the diamond blade surface can be achieved by using a temperature of 235° C. for approx. 1 hour.

In respect of diamond there is an additional difficulty in chemically bonding the coating material to its surface. This is due to the fact that in general a diamond surface does not have hydroxyl groups attached to its surface. Methods of applying a hydroxyl-coated surface are therefore part of various embodiments of the invention. One such method achieves this by immersing the diamond surface in a bath of molten alkali hydroxide, such as sodium hydroxide or potassium hydroxide or mixtures of these with sodium- or potassium nitrate for periods of up to one hour. Another and preferred method is the application of a microwave discharge in water vapour to the diamond surface. This dissociates water molecules and forms OH radical groups in vapour form, which can attach to the diamond surface. Other methods include application of an interfacial layer, such as titanium (Ti), chromium (Cr) or Silicon (Si). The layer can be hydroxyl terminated by immersion in dilute NaOH. It is also possible to attach the fluoroaliphatic silyl ether to the metal surface directly by dipping the freshly coated surface into the coating liquid.

Formation of a hydroxyl-terminated Si layer can also be achieved by immersing the diamond blade in a dilute (approx. 10%) solution of NaOH in water for approx. 3 minutes at approx. 90-100° C., followed by rinsing in deionized water, dipping in a concentrated (>20%) solution of HCl in water, rinsing again in deionized water, rinsing in ethanol and finally isopropanol and then allowing the blade to dry. After this step the blade is immersed in the coating liquid and the coating is applied as described above.

The preferred interfacial layer for attaching a layer of coating molecules to a diamond surface has been to pre-coat the surface of the diamond with a thin layer of silicon (Si). This layer, which is typically less than 50 nm thick forms a chemical bond with the diamond by the formation of SiC. A larger thickness of the Si layer is disadvantageous as it will result in a reduced transmission of the infrared radiation out of the blade and concomitant absorption of the radiation in the blade, leading to a reduced cauterising effect in the tissue and/or heating of the blade and extra sticking of tissue or blood to the blade. For applications where light is not required to exit the Si layer the layer may be applied thicker or another interfacial layer may be applied.

The surfaces to which this process may be applied are formed of hard, transparent crystalline material. Typically this material is natural, monocrystalline synthetic or polycrystalline synthetic diamond or sapphire. However, other materials could also be used such as hard crystalline simple oxides such as zirconia (Zro₂), yttria (Y₂0₃), garnets, most notably YttriumAluminumGarnet, LutetiumAluminumGarnet, vanadates and aluminumoxides (such as YttriumAluminumOxide.) Other hard infrared transparent crystals which may also be appropriate for the process are, orthosilicates.

The method which forms the subject of various embodiments of the invention can be applied to a wide range of cutting instruments operating in a range of laser wavelengths, such as those which are described in South African provisional patent application no. 99/4256.

In an example of the invention, a diamond window suitable for use in a surgical operation such as prostrate cancer is provided. A surface of the diamond window was placed on a glass plate in a suitable microwave chamber. Water vapour was introduced (the residual gases being nitrogen and oxygen) and subjected to microwave energy at a discharge frequency of 2.45 GHz. The discharge duration was 30 minutes. This caused the water molecules to dissociate forming OH radical groups which deposited on the surfaces of the diamond window and attached to these surfaces.

The thus treated diamond window then had a protective layer of fluorine atoms applied to it using fluoroaliphatic silyl ether in the manner described above. The coating had a thickness of about 300 nanometers.

What is claimed is:

1. A method of forming a protective layer of fluorine atoms on a surface of a surgical instrument comprising the step of:
immersing the surface into a solution of a fluoroaliphatic silyl ether.

2. The method according to claim 1 further including the step of:
curing the protective layer at a temperature in excess of 200° C.

3. The method according to claim 1 further including the step of forming a hydroxyl terminated surface on the surface before immersion of the surface into a solution of a fluoroaliphatic silyl ether.

4. The method according to claim 3 wherein the hydroxyl terminated surface is formed by the microwave discharge of water vapour.

5. The method according to claim 4 wherein the surface is a diamond surface and the microwave discharge dissociates water molecules to form OH radical groups in the vapour form which attach to the diamond surface.

6. The method according to claim 1 further including the step of forming an intermediate silicon (Si) layer on the surface of the surgical instrument prior to immersion of the blade into a solution of a fluoroaliphatic silyl ether.

7. The method according to claim 6, wherein the Si layer has a thickness less than 50 nm.

8. The method according to claim 1 wherein the surface is a surface of a surgical instrument suitable for a single application.

9. The method according to claim 8 wherein the surface is a surface of a window through which laser or like radiation passes, in use.

10. The method according to claim 1 wherein the surface is a surface of a cutting blade.

11. The method according to claim 1 wherein the protective layer has a thickness of no more than 700 nanometres.

12. A method of forming a protective layer of fluorine atoms of no more than 700 nanometres on a surface of a surgical instrument comprising the steps of:
forming a hydroxyl terminated surface on the surface; and
immersing the thus treated surface into a solution of a fluoroaliphatic silyl ether.

13. The method of claim 12 wherein the hydroxyl terminated surface is formed by microwave discharge of water vapour.

14. The method according to claim 13 wherein the surface is a diamond surface and the microwave discharge dissociates water molecules to form OH radical groups in the vapour form which attached to the diamond surface.
